(19) United States
(12) Patent Application Publication
(10) Pub. No.: US 2008/0014283 A1
(43) Pub. Date: Jan. 17, 2008

(54) TREATMENT METHODOLOGIES FOR PREVENTING REFLUX INDUCED ADENOCARCINOMA OF THE ESOPHAGUS

(76) Inventor: Parakrama Chandrasoma, Pasadena, CA (US)

Correspondence Address: KAUTH , POMEROY , PECK & BAILEY ,LLP, P.O. BOX 19152, IRVINE, CA, 92623, US

(21) Appl. No.: 11/844,218

(22) Filed: Aug. 23, 2007

Related U.S. Application Data
(63) Continuation-in-part of application No. 11/669,059, filed on Jan. 30, 2007.
(60) Provisional application No. 60/763,649, filed on Jan. 30, 2006.

(51) Int. Cl. A61K 33/00; A61P 1/00; C12Q 1/02

(52) U.S. Cl. 424/600; 435/29

(57) ABSTRACT

Medications for the prevention and treatment of the progression of gastroesophageal reflux disease to adenocarcinoma, and more specifically to medications that operate in accordance with a new mechanistic understanding of the sequence of changes whereby gastroesophageal reflux disease progresses to adenocarcinoma are provided. The medications for the prevention and treatment of reflux-induced adenocarcinoma are based on (1) recognizing cardiac mucosa as an abnormal epithelium that should be a target of treatment, (2) preventing cardiac mucosa from progressing to intestinal metaplasia (Barrett esophagus), and (3) promoting the conversion of cardiac mucosa to oxyntocardiac mucosa, which is recognized by the invention as being a benign epithelium that does not progress to intestinal metaplasia (Barrett esophagus) and adenocarcinoma. In patients who already have intestinal metaplasia at the time of detection, the medications for prevention and treatment require a preliminary step whereby the intestinal metaplasia is reverted to cardiac mucosa before it can be induced to convert to the benign oxyntocardiac mucosa.

### CROSS-REFERENCE TO RELATED APPLICATIONS

The current application claims is a continuation-in-part of U.S. application Ser. No. 11/669,059, filed Jan. 30, 2007, which claims priority to U.S. Provisional Application No. 60/763,649, filed Jan. 30, 2006, the disclosure of both applications are incorporated herein by reference.

### FIELD OF THE INVENTION

The present invention relates in general to a treatment for gastroesophageal reflux disease, and more specifically to medications that operate through a novel mechanistic understanding of gastroesophageal reflux disease to prevent reflux-induced adenocarcinoma of the esophagus.

### BACKGROUND OF THE INVENTION

Few diseases have caused as much confusion and controversy as the columnar lined esophagus that bears the name of Norman Barrett.

Barrett began its eventful history in 1950 when its namesake proclaimed that it did not exist, declaring that the esophagus should be defined as “that part of the foregut, which is lined by squamous epithelium.” (Barrett N R. Chronic peptic ulcer of the oesophagus and “oesophagitis”. Br J Surg 1950:38:175-182, the disclosure of which is incorporated herein by reference.) Allison, who described reflux esophagitis in 1948 and columnar lined esophagus in 1953, in contrast got it almost perfectly right on his first attempt. (See, e.g., Allison P R. Peptic ulcer of the esophagus. Thorax 1948:3:20-42; and Allison P R, Johnstone A S. The oesophagus lined with gastric mucous membrane. Thorax 1953:8:87-101, the disclosures of which are incorporated herein by reference.) He accurately interposed a columnar lined distal esophagus between the squamo-columnar junction and the proximal stomach. In 1957, Barrett's reversed his opinion and agreed with Allison that the tubular structure distal to the squamo-columnar junction was a columnar lined esophagus (Barrett N R. The lower esophagus lined by columnar epithelium. Surgery 1957:41:881-894, the disclosure of which is incorporated herein by reference.) When these classical papers are reviewed, it is incomprehensible that this entity came to be known as Barrett esophagus and not Allison esophagus.

A little recognized fact is that when Norman Barrett reversed himself in 1957, the only histologic definition of the esophagus that has ever existed disappeared. Although the definition was wrong, Barrett's idea was correct: without a histologic definition of the esophagus, gastroesophageal junction and stomach, confusion would surely reign. Indeed, confusion has certainly reined supreme in the 50+ years after Allison. To date, pathologists have no accepted method of accurately defining the demarcation between the esophagus and stomach. Instead, most of us depend on the endoscopist to tell us the location of the gastroesophageal junction and use this information to decide whether a given biopsy sample is from the esophagus or the stomach.

Confusion regarding the diagnosis of Barrett esophagus exists because of a false dogma that cardiac mucosa is normally present in the gastroesophageal junctional region. In fact, this view of what constitutes the normal and healthy functioning of the esophagus is generally well-accepted by the majority of physicians. The conventional failure to properly characterize these epithelia in patients with reflux disease and the failure to recognize that they are abnormal has resulted in a failure of management of patients with reflux disease. This failure has partly been responsible for the explosion in the incidence in reflux-induced adenocarcinoma of the esophagus in the past thirty years. Esophageal adenocarcinoma is the most rapidly increasing cancer type in the United States.

Recent studies of thousands of biopsies of the esophagus in patients with reflux disease over the past fifteen years have provided strong evidence that the effect of the refluxed fluid that passes from the stomach into the esophagus during gastroesophageal reflux disease and has varied effects on the different epithelial types in the esophagus. The epithelial types identified in these studies include stratified squamous epithelium, which is the normal epithelial lining of the esophagus, and three types of metaplastic columnar epithelia, including cardiac mucosa, cardiac mucosa with intestinal metaplasia, and oxyntocardiac mucosa. These columnar epithelial types are indistinguishable by endoscopy; however, they are diagnosed by easily applicable histologic criteria in endoscopic biopsies as reported in U.S. patent application Ser. No. 11/669,059, the disclosure of which is incorporated herein by reference.

Accordingly, new treatments and medications are needed that can take advantage of this new understanding of Barrett esophagus to provide an effective methodology for preventing reflux induced adenocarcinoma at an early stage of its development.

### SUMMARY OF THE INVENTION

The present invention is directed generally to novel medications for the prevention and treatment of gastroesophageal reflux disease, and more specifically to medications that operate in accordance with a new mechanistic understanding of gastroesophageal reflux disease to prevent reflux induced adenocarcinoma of the esophagus. The medications of the current invention are based on a fundamental understanding that the only normal epithelia in the esophagus and proximal stomach are squamous epithelium and gastric oxyntic mucosa.

In another embodiment, the current invention is also directed to a drug discovery methodology for developing additional treatments for the prevention and treatment of gastroesophageal reflux disease.

The above-mentioned and other features of this invention and the manner of obtaining and using them will become more apparent, and will be best understood, by reference to the following description, taken in conjunction with the accompanying drawings. The drawings depict only typical embodiments of the invention and do not therefore limit its scope.

### BRIEF DESCRIPTION OF THE FIGURES

FIG. 1, provides photographic illustrations of squamous epithelium, wherein A: shows a normal squamous epithelium with tight junctions and no separation between squamous cells in the epithelium; and B & C: show reflux-damaged squamous epithelium showing separation of the squamous epithelial cells, making it more porous and permitting entry of luminal molecules.

FIG. 2, provides photographic illustrations of squamous epithelium, wherein A: shows columnar metaplasia of stratified squamous epithelium; and B: Shows the transition from stratified squamous (left) to columnar metaplastic (cardiac) mucosa (right).

FIG. 3, provides photographic illustrations of metaplastic cardiac mucosa in the esophagus, wherein A: shows that the epithelium consists of a surface epithelial layer and a foveolar pit composed only of mucous cells; and B: cardiac mucosa stained by immunoperoxidase technique for Ki67 (MIB-1).

FIG. 4, provides a schematic diagram showing the reflux to adenocarcinoma sequence.

FIG. 5, provides a pictorial illustration of the evolution of cardiac mucosa in the esophagus, wherein A: shows the conversion of cardiac mucosa to oxyntocardiac mucosa; and B: shows the conversion of cardiac mucosa to intestinal metaplastic (Barrett's) epithelium.

FIG. 6, provides a graph of pH gradient changes in the esophagus in gastroesophageal reflux in patients with and without acid suppressive drug therapy.

FIG. 7, provides a graph of the change in pH gradient caused by acid suppression in two patients, one with mild reflux (left) and one with severe reflux (right).

FIG. 8, provides a schematic comparison of the distribution of intestinal metaplasia (blue), cardiac mucosa (black) and oxyntocardiac mucosa (red) at two periods in a patient's history.

FIG. 9, provides a series of schematics showing four patients with different and constant levels of reflux: (A) a patient without reflux, (B) a patient with mild reflux, (C) a patient with moderate reflux, and (D) a patient with severe reflux with a long segment of columnar lined esophagus.

FIG. 10, provides: (A) a photograph of a standardized biopsy protocol in patients with columnar lined esophagus, and (B) a schematic showing the different columnar epithelial types in each biopsy.

FIG. 11, provides: (A) a schematic showing the progression of reflux-induced columnar epithelium to adenocarcinoma through cardiac mucosa (black) and intestinal metaplasia (blue), and (B) a regression and “cure” induced by the new drugs results in conversion of all cardiac and intestinal epithelia into oxyntocardiac mucosa (red).

FIG. 12, provides a photographic illustration of a postulated experimental system that would be utilized to detect the conversion of cardiac epithelial cells to intestinal metaplasia in vitro.

FIG. 13, provides a photographic illustration of a postulated experimental system that would be utilized to detect the conversion of intestinal metaplastic epithelium to cardiac mucosa in vitro.

FIG. 14, provides a photographic illustration of a postulated experimental system that would be utilized to detect the conversion of cardiac to oxyntocardiac mucosa in vitro.

FIG. 15, provides a schematic diagram illustrating the effects of the pH gradient created by gastroesophageal reflux in different components of the esophageal epithelium.

FIG. 16, provides a schematic diagram illustrating the effects of the pH changes induced in the esophagus with acid suppression.

FIG. 17, provides a schematic diagram showing a sequence of the changes expected from strong acidification of the entire segment of the reflux damaged esophagus.

FIG. 18, provides a schematic diagram of one exemplary embodiment of a drug in accordance with the current invention designed to prevent reflux-induced esophageal adenocarcinoma.

FIG. 19, provides a schematic diagram showing the effects of the administration of an exemplary embodiment of the drug of the current invention when introduced orally in a dosage sufficient to produce an adequate concentration in gastric juice, which would result in the drug being delivered to the entire length of affected esophagus when the patient has episodes of gastroesophageal reflux.

FIG. 20, provides a schematic diagram showing the effects of the administration of an exemplary embodiment of the drug of the current invention when used in combination with acid suppressive drugs.

### DETAILED DESCRIPTION OF THE INVENTION

The present invention is directed generally to novel medications for the prevention and treatment of gastroesophageal reflux disease, and more specifically to medications that operate in accordance with a new mechanistic understanding of gastroesophageal reflux disease to prevent reflux induced adenocarcinoma of the esophagus. The medications of the current invention are based on a novel understanding of gastroesophageal reflux disease that recognizes that the only normal epithelia in the esophagus and proximal stomach are squamous epithelium and gastric oxyntic mucosa.

This application is divided into two sections. First, the application provides a review of the new disease mechanism, hereinafter “reflux carditis”, and diagnostic methodologies previously described in U.S. patent application Ser. No. 11/669,059, the disclosure of which is incorporated herein by reference. The application then describes new treatments designed to treat reflux carditis and thereby prevent reflux-induced adenocarcinoma of the esophagus.

The Mechanisms of Gastroesophagael Reflux

Despite the vast sums of money being spent on research into treating reflux disease, very little progress has been made in preventing the most serious consequence of the disorder, namely adenocarcinoma. It has been surprisingly discovered that the reason for this continued failure lies in a fundamental misunderstanding of the basic causes of gastroesophageal reflux disease, and the concomitant misdirection of research. Part of the reason for this lack of understanding of the cellular basis of reflux disease is that there is little cross-pollination between the gastroenterologists and surgeons in the field, who have absolutely no training in microscopic pathology, and the pathology establishment. Spechler stated the problem well in 1997: “Conceptually, (columnar lined esophagus) can be defined simply as the condition in which the stratified squamous epithelium of the distal esophagus is replaced by a metaplastic, columnar epithelium. Practically, it can be difficult to apply this conceptual definition in clinical situations because of difficulties in delimiting the distal esophagus and the proximal stomach and in distinguishing normal from metaplastic columnar epithelium on the basis of endoscopic appearance.” (Spechler S J., “The columnar lined esophagus.” Gastroenterol. Clin. N. Amer. 1997:26:455-465, the disclosure of which is incorporated herein by reference.) Although based on a faulty understanding of the mechanism of reflux disease, this failure is directly caused by the misdiagnosis of patients during conventional Barrett esophagus surveillance programs. As a result, there is very little specific effort being made to address the approximately 14,000 patients who die every year from esophageal adenocarcinoma.

Using the novel diagnostic techniques disclosed in U.S. patent application Ser. No. 11/669,059, the disclosure of which is incorporated herein by reference, it is now possible to accurately detect the presence of reflux carditis in patients at an early stage. In short, the diagnostic techniques use the presence of metaplastic columnar epithelium to diagnose reflux disease, and uses its length to define the severity of reflux disease. The current application addresses the causes of this reflux carditis, and in turn provides novel treatment methodologies for preventing the mechanistic triggers that cause reflux carditis and ultimately lead to esophageal adenocarcinoma.

The Effect Of Refluxed Acid On Squamous Epithelium

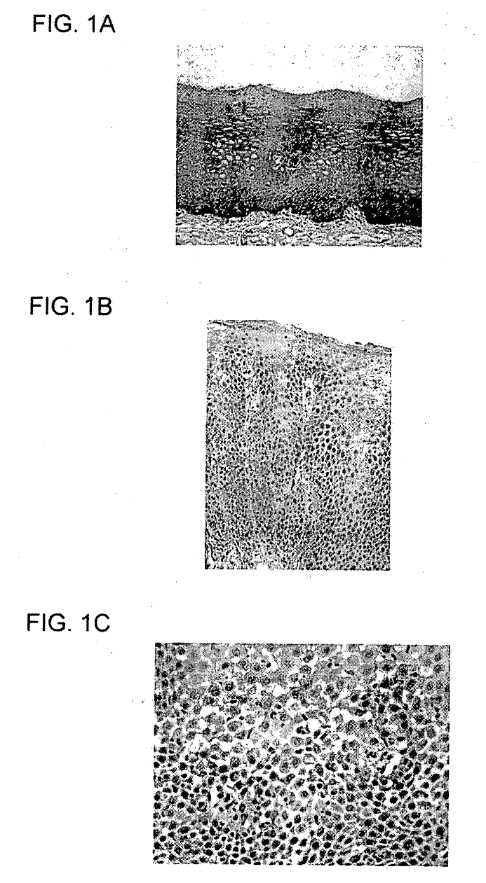

During the onset of reflux carditis, normal squamous epithelium of the esophagus is damaged by acid (free H+ ions). Specifically, acid induced damage is produced by introduction of an over-concentration of H+ ions into squamous cells, and results in separation of the tight junctions of the stratified squamous epithelium and the development of “dilated intercellular spaces”. See, for example, FIG. 1, which provides a comparison of normal squamous epithelium and reflux-damaged squamous epithelium.

As shown, in normal epithelium (FIG. 1A), the tight junctions and lack of separation between squamous cells in the epithelium makes the epithelium impermeable to the entry of luminal molecules. However, in reflux-damaged squamous (FIGS. 1A & 1B), the separations in the acid damaged epithelium allows the luminal molecules to pass into the epithelium through the now separated tight junctions. The size of the particles that are permitted to penetrate the squamous epithelium, and the depth to which the molecules can penetrate are dependent on the severity of damage. As the damage becomes more severe, molecules of increasing size move into increasingly deeper regions of the squamous epithelium. In vitro experiments have shown that severely acid-damaged squamous epithelium permits molecules up to 20 kD to penetrate the stratified squamous epithelium and reach the proliferating cell pool in the basal layers³.

The pain of gastroesophageal reflux disease is produced when penetration of luminal molecules reaches the pain-sensitive sensory nerve endings that are situated in the mid-region of the squamous epithelium of the esophagus. Stimulation of these nerve endings results in heartburn. Acid is the most powerful stimulant of these nerve endings, although evidence exists that non-acid molecules can also be responsible for some pain. The final effect of acid on the squamous epithelium is erosion and ulceration of the epithelium resulting in denudation of the surface. This in turn causes erosive esophagitis.

It is important that the dilated intercellular spaces that permit the entry of luminal molecules into the deep regions of the epithelium are relatively small. Drugs that are developed that have a large size are not likely to pass into the epithelium (FIG. 1C). Even the reflux-damaged squamous epithelium will permit the entry of only moderate sized molecules. As such, any drug that has a molecular size greater than the space between the separated intercellular spaces will not enter the squamous epithelium and is separated from the stem and/or proliferative cells in the basal region of the epithelium.

Effects Of Non-Acid Molecules On Squamous Epithelium

Non-acid molecules in the esophageal lumen in patients with reflux include molecules ingested as food and drink, molecules secreted by gastric mucosa, and in patients with duodenogastric reflux, molecules secreted by the duodenum, bile and pancreatic juice. In a patient with acid-induced increased permeability of the squamous epithelium, all the molecules that are within the size range to pass between the dilated intercellular spaces can enter and penetrate the squamous epithelium. These non-acid molecules can also potentiate the cellular damage in squamous epithelium caused by acid.

The penetration of non-acid molecules of the refluxate deep into the squamous epithelium permits interactions between these molecules and the proliferating pool of cells in the basal region as well as the stem cells in the basal layer. This is the mechanism that in turn leads to columnar metaplasia of the esophagus.

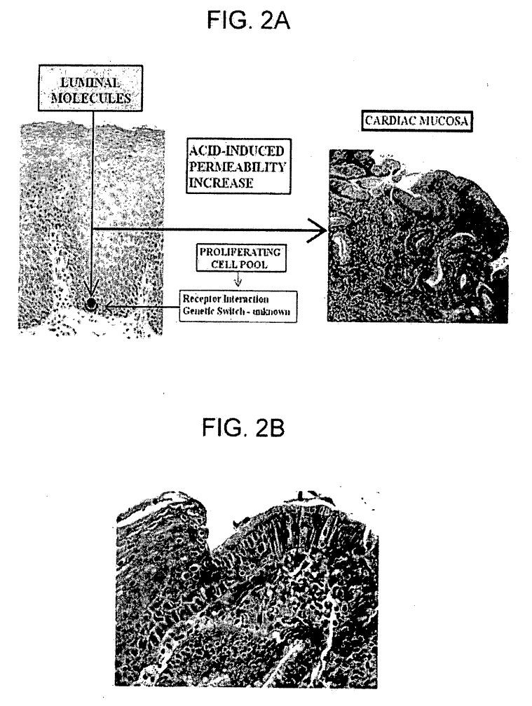

FIG. 2 provides a photograph demonstration of this transformation. As shown in FIG. 2A, first refluxate molecules penetrate the damaged squamous epithelium and interact with the proliferating and/or stem cell pool in the basal region. This causes a switch of the genetic signal that controls differentiation of the proliferating and/or stem cell (FIG. 2A). In turn, FIG. 2B shows the transition from stratified squamous (left) to columnar metaplastic (cardiac) mucosa (right). Note the cleavage plane in the squamous epithelium immediately above the basal layer, this change represents impending columnar metaplasia.

Whereas normally the genetic signal dictates squamous differentiation of the proliferating cells, the genetic switch suppresses the squamous signal and replaces it with a columnar differentiating signal. Although the exact nature of the luminal molecule and the gene that causes the normal squamous differentiation on the stem cell in the esophagus is unknown. There is evidence that the gene that directs initial columnar (cardiac) metaplasia in the esophagus is related to the Homeobox genes, and may include Cdx1 and Cdx2. Accordingly, in one embodiment of the current invention this gene is the target of preventative medication.

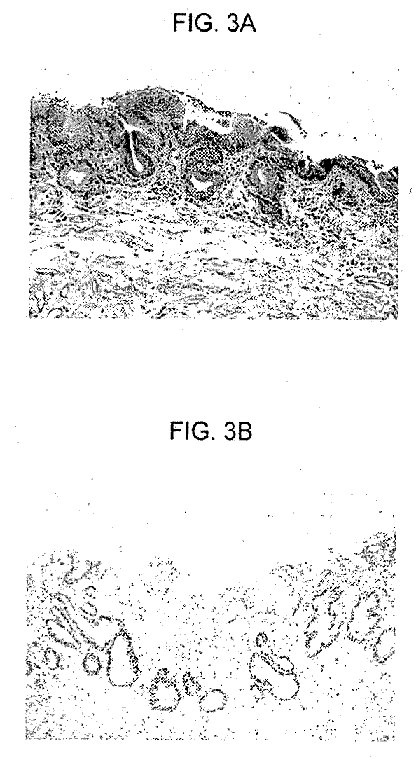

The cardiac mucosa that results from columnar metaplasia is an epithelium composed of mucous cells only, without any specialized cells. It has a surface epithelium and a foveolar pit, as shown in FIG. 3A. In turn, the proliferating pool of cells that include the stem cells reside in the deep foveolar region of the epithelium, as shown in FIG. 3B. These figures show positivity restricted to the deep foveolar region, indicating that this is the location of the stem and/or proliferating cell pool in the epithelium. The stem and/or proliferating cell pool is the site of molecular interactions that cause genetic switches. The luminal molecules (including drugs) gain access to the stem and/or proliferating cell pool through the opening of the foveolar pit. Accordingly, luminal molecules of large size, including any drugs that are present in the refluxate, can easily access the proliferating stem cells because of the large size of the foveolar pit.

Evolution Of Cardiac Mucosa In The Esophagus

Studies indicate that the cardiac mucosa, discussed above, that is produced in the esophagus as an early manifestation of reflux disease progresses in one of two directions when a second genetic switch occurs. In the first direction, the second genetic switch results in oxyntocardiac mucosa with the development of parietal cells. In the second direction, the second genetic switch results in intestinal metaplasia with the development of colonic-type goblet cells. Evidence suggests that these two genetic switches are mutually exclusive. Oxyntocardiac mucosa does not develop goblet cells and intestinal metaplasia does not develop parietal cells. Of the two types of mucosa, only the intestinal metaplasia progresses to cancer.

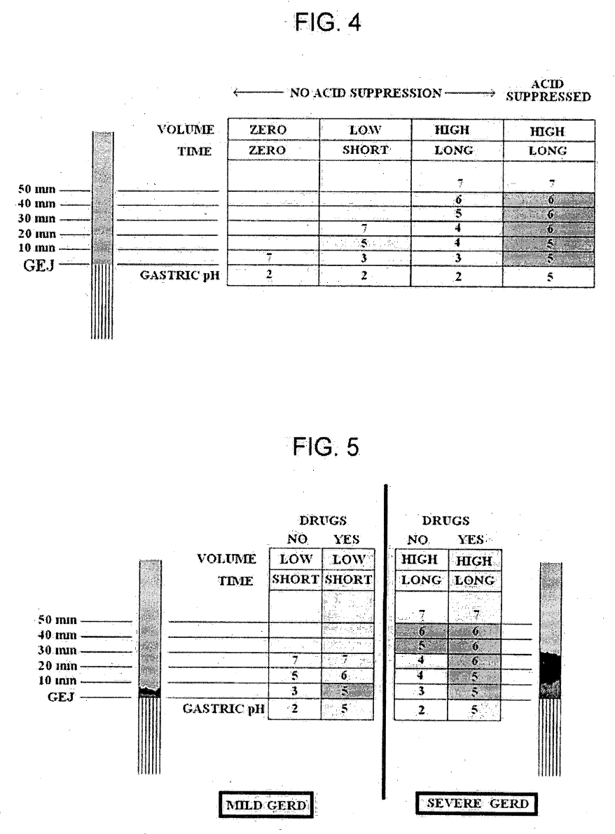

A schematic diagram of this reflux to adenocarcinoma sequence is provided in FIG. 4. As shown, the pathway to oxyntocardiac mucosa results in a stable columnar epithelium that is relatively insensitive to pain and does not progress to cancer, while the pathway to intestinal metaplasia results in a premalignant epithelium that progresses to cancer. It is important to recognize that these two genetic switches are not sequential. The development of oxyntocardiac mucosa takes the esophageal epithelium outside the reflux-adenocarcinoma sequence and this epithelium is not at risk for cancer. Accordingly, in one embodiment, the preventative treatment methodology of the current invention is directed to controlling the direction of the mucosa development in a patient at risk of developing reflux carditis. These switches are discussed in greater detail below.

The first direction of the second genetic switch results in the transformation of cardiac mucosa into oxyntocardiac mucosa. This is a columnar epithelium in the esophagus that has developed glands and developed the ability to differentiate into parietal (oxyntic) cells. When cardiac mucosa transforms to oxyntocardiac mucosa, it appears to preclude the progression to intestinal metaplasia. As such, the conversion of cardiac mucosa to oxyntocardiac mucosa represents an end-point that prevents progression to cancer.

FIG. 5 provides an illustration of the evolution of cardiac mucosa in the esophagus. First, further metaplastic events in cardiac mucosa result from luminal molecules interacting with the stem and/or proliferating cells in the cardiac mucosa, causing genetic switches that direct the new types of columnar epithelia. As shown in FIG. 5A, first there is a conversion of cardiac mucosa to oxyntocardiac mucosa. This occurs in a strong acid milieu of the most distal part of the columnar lined segment in the esophagus in patients with reflux disease. Recapitulating this environment with drugs can promote this change. As shown in FIG. 5B, conversion of cardiac mucosa to intestinal metaplastic (Barrett's) epithelium occurs in the more proximal region of the columnar lined segment where the milieu is less acid in the pH 4-6 range. In turn, prevention of this milieu by drugs can prevent intestinal metaplasia in cardiac mucosa.

Evidence suggests that the metaplastic switch from cardiac mucosa to oxyntocardiac mucosa is associated with the expression of a suppressed differentiating gene complex that includes the Sonic Hedgehog gene.

The other direction of the second genetic switch results in the transformation of cardiac mucosa to intestinal metaplasia by the development of goblet cells in the epithelium which previously contained only mucous cells, as shown in FIG. 5B. This second metaplastic event effectively defines Barrett's esophagus.

Barrett's esophagus is a recognized premalignant disease of the esophagus. At the present time, it is estimated to have a cancer risk of 0.5% per year. Given the fact that there are approximately 5 million adults in the USA with Barrett's esophagus, this risk translates to the 20,000 to 25,000 people who are estimated to develop reflux-induced adenocarcinoma in the USA in 2007. Barrett's esophagus has been shown to progress through low grade dysplasia and high grade dysplasia to invasive adenocarcinoma. At the present time, invasive adenocarcinoma of the esophagus is one of the most lethal cancer types with an overall mortality in the 85% range.

Evidence suggests that the second metaplastic switch from cardiac mucosa to intestinal metaplasia is associated with the expression of the suppressed CDX homeobox gene complex, notably CDX2. It is to be emphasized that the presence of intestinal metaplasia in the esophagus is necessary for effective carcinogenesis. All other epithelia found in the esophagus, including squamous epithelium, cardiac mucosa and oxyntocardiac mucosa are not susceptible to action by carcinogens in the refluxate. If a patient has no intestinal metaplasia, there is no risk of cancer. However, if a patient has cardiac mucosa, there is the possibility that a future risk will emerge if that patient develops intestinal metaplasia.

The Genetic Switch

As discussed above, the proposed mechanism of metaplasia of the esophageal epithelium in reflux disease involves genetic switches. Three genetic switches have been discussed. Two are sequential in the reflux to adenocarcinoma sequence and results first in the conversion of stratified squamous epithelium to cardiac mucosa and then the conversion of cardiac mucosa to intestinal metaplasia, as shown in FIG. 4. The third genetic switch, which converts cardiac mucosa to oxyntocardiac mucosa, removes the patient from the sequence whereby reflux results in cancer.

A genetic switch is defined as a replacement of an active genetic signal that determines cell differentiation with a previously suppressed genetic signal. The old signal is superceded by the new signal, which directs cell differentiation in a new direction. In the present case, the first switch causes the esophageal stem cell to stop differentiating into squamous epithelium and causes it to differentiate into columnar (cardiac) mucosa, while the second genetic signal causes cardiac mucosa to develop either parietal cells (oxyntocardiac mucosa) or goblet cells (intestinal metaplasia).

The genes involved in genetic switches are normally present in the genome. They are normal genes that direct differentiation in other parts of the foregut, but are normally suppressed in the adult esophageal stem cell. The gene that directs cardiac mucosal differentiation was probably expressed in the esophageal stem cell during fetal life when it transiently caused the esophageal mucosa to be columnar. The Sonic Hedgehog gene that directs oxyntocardiac mucosa is expressed in the normal stomach. The CDX2 gene that is involved in intestinal metaplasia in cardiac mucosa is normally expressed in the colon.

Because genetic switches involve activation and suppression of genes that are normally present in the genome, they can be readily manipulated. Effects of their activation and suppression are instantaneously manifested. These are very different genetic changes than those produced during carcinogenesis, in which permanent and irreversible genetic mutations result. These are much more difficult to reverse and have a long lag phase before they express themselves phenotypically. Accordingly, in one embodiment of the invention, as will be discussed further below, a treatment is designed to control this genetic switch, and therefore control the direction of evolution of cardiac mucosa towards oxyntocardiac mucosa and away from intestinal metaplasia in the early stage of the disease to prevent cancer in patients with reflux disease.

Effects of Acid Suppressive Drugs on Reflux Carditis

At the present time acid suppressive drugs represent the only effective drug treatment of reflux disease. Accordingly, the majority of pharmaceutical research in reflux disease at the present time is directed towards producing better and more effective acid suppressive agents.

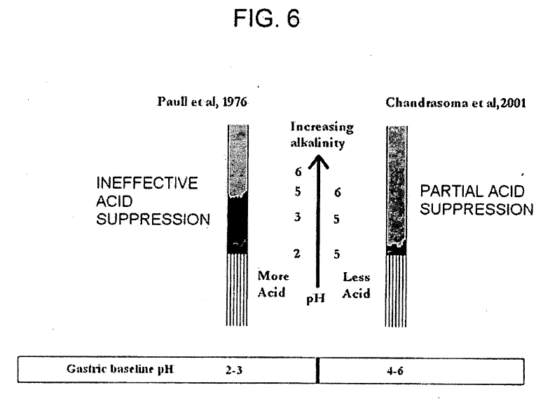

Acid suppressive drugs are designed to prevent native acid secretion by gastric parietal cells. When used in effective dosage, they increase the baseline pH of the gastric juice. When reflux of this more alkaline gastric juice into the esophagus occurs, the resulting pH gradient in the esophagus is dramatically altered, as shown in FIG. 6, which shows the pH gradient changes in the esophagus in gastroesophageal reflux in patients with and without acid suppressive drug therapy.

In the left column of FIG. 6 is a normal patient without reflux. As shown, the lower esophageal sphincter acts as a barrier that effectively separates the neutral esophageal pH from the strongly acid pH of the stomach. In the next column of FIG. 6 is shown a patient with mild reflux, the reflux episode is low volume and causes a pH gradient over a short segment of the esophagus (shown here as 20 mm). The exposure time is short because the volume of reflux is small in mild reflux. In the next column of FIG. 6 is a patient with severe reflux with a high volume and long exposure. As shown, the pH gradient has shifted proximally with the more distal esophagus being more strongly acid. In the right column of FIG. 6 is the same patient with severe reflux on acid suppressive drugs. As shown, the pH gradient has changed with the entire length exposed to the higher pH because the drugs have caused the gastric pH to become less acid.

In summary, FIG. 6 illustrates that whereas before acid suppression the esophageal pH milieu tended to be strongly acidic in the distal region with a gradient of pH till it reached neutrality at the proximal limit of the reflux, the milieu is much more alkaline throughout the esophagus in the patient who is on acid suppressive drugs.

To understand how this pH change alters the course of reflux carditis it is important to revisit the evolution of mucosa in a patient with reflux carditis. First, the distribution of the three different epithelial types in columnar lined esophagus is constant⁴. Oxyntocardiac mucosa occurs in the most distal region, and when present, intestinal metaplasia favors the most proximal region. When this fact is correlated with the pH gradient that is set up in the esophagus during a reflux episode, it suggests that the conversion of cardiac to oxyntocardiac mucosa is favored by a strong acid (pH 1-3) milieu and intestinal metaplasia by a higher pH (4-6) milieu.

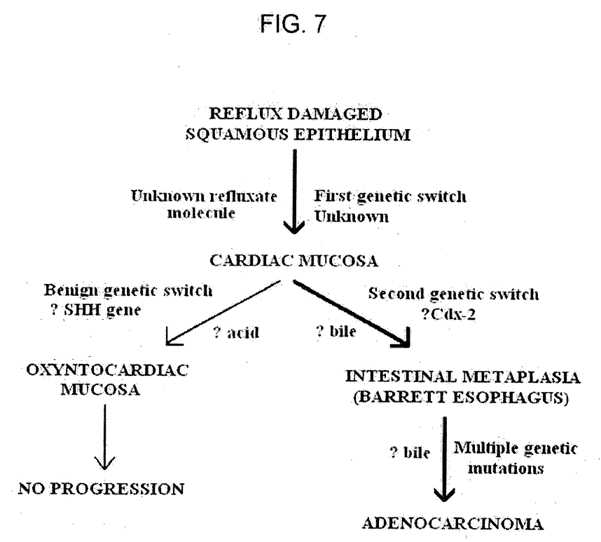

This is illustrated by FIG. 7, which shows the effect of the change in pH gradient caused by acid suppression in two patients. In the patient on the left with mild reflux, the columnar lined segment is short. The mild reflux creates a gradient, which has a short time exposure plus a strong acid environment throughout the columnar lined segment (black=cardiac mucosa; red=oxyntocardiac mucosa). In the patient on the right with severe reflux, the length of columnar metaplasia is greater and has an acid (pH 4 or less) environment. There is little risk of intestinal metaplasia in this situation. When acid suppressive drugs are used, the baseline gastric pH becomes higher and the gradient in the esophagus with reflux shifts accordingly. The risk of intestinal metaplasia increases dramatically.

Accordingly, the metaplastic event in oxyntocardiac mucosa is either caused by hydrogen ions, other anions, or a non-acid molecule in the refluxate whose interaction with the proliferating and/or stem cells in cardiac mucosa is favored by a low pH (strong acid) environment. Likewise, the metaplastic event in intestinal metaplasia is probably caused by a non-acid molecule in the refluxate whose interaction with the proliferating and/or stem cells in cardiac mucosa is favored by the 4-6 pH range (weak acid environment) and inhibited or reversed by a strong acidic milieu in the pH 1-3 range. This is the same milieu strong acid milieu that promotes the conversion of cardiac mucosa to oxyntocardiac mucosa.

As discussed above, acid suppressive drugs act by suppressing acid secretion by the stomach and therefore cause a reduction in the baseline gastric pH. When reflux occurs in the patient who is on acid suppressive drug therapy, the higher gastric baseline pH (commonly in the 4-6 range) is reflected in a pH gradient in the esophagus that is less acid than it would have been if the patient had not been acid suppressed. As such, acid suppressive therapy favors the cardiac mucosa to progress to intestinal metaplasia along a greater length of the columnar lined esophageal segment. The 4-6 pH also prevents cardiac mucosa from transforming into oxyntocardiac mucosa. In short, these two events occurring in these early metaplastic biologic reactions in the esophagus tend to increase the risk of adenocarcinoma.

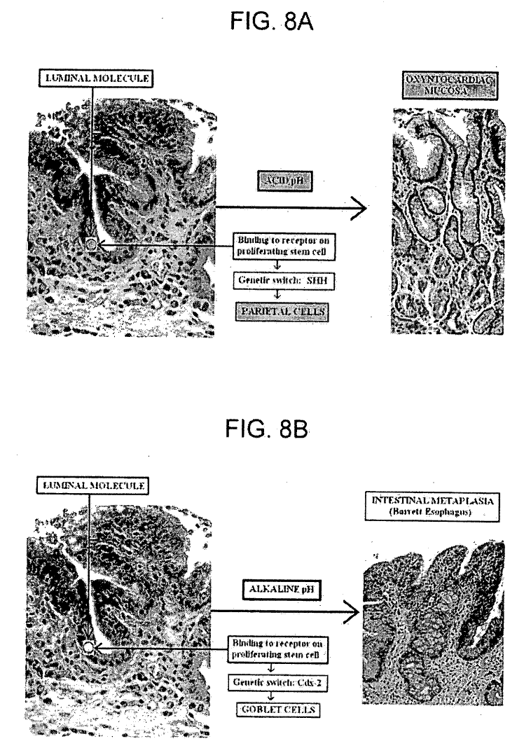

The role of acid suppressive drugs in promoting the risk of adenocarcinoma is illustrate in FIG. 8, which provides a comparison of the distribution of intestinal metaplasia (blue), cardiac mucosa (black) and oxyntocardiac mucosa (red) at two periods in history. In 1976 (FIG. 8, left) intestinal metaplasia was less common and occupied only the more proximal region of the columnar lined segment. This contrasts with 2003 (FIG. 8, right) where equivalent lengths of columnar lined segments had much more intestinal metaplasia extending much lower in the esophagus. This is correlated with the effectiveness of acid suppression and the change in the pH gradient in the esophagus during a reflux episode.

In summary, the change in esophageal pH promoted by acid suppressive drugs has favored a shift in the amount of intestinal metaplasia that is produced. In the past, before powerful acid suppressive drugs were developed, intestinal metaplasia was uncommon and limited to the most proximal part of the columnar lined segment in the esophagus. At present⁶, intestinal metaplasia occurs much more distally. Accordingly, it is likely that the increase in the incidence of intestinal metaplasia (or Barrett esophagus) in the past three decades has been the result of the change in the esophageal pH gradient resulting from increasingly effective acid suppressive therapy. The increased incidence of intestinal metaplasia in turn would be expected to result in an increased incidence of reflux-induced cancer, which has also occurred.

In short, acid suppressive therapy by itself does not stop reflux⁷. Rather, gastroesophageal reflux continues because it is a manifestation of an abnormality of the lower esophageal sphincter, whose deficiency is not addressed in any substantial manner by acid suppressive therapy. The fact that gastroesophageal reflux continues in patients on acid suppressive therapy has been shown by impedance technology. This means that the esophageal epithelium in patient with reflux induced changes continue to be bombarded by non-acid molecules in the refluxate in a milieu of a higher pH while their symptoms are controlled with acid suppressive drugs.

Neither do acid suppressive drugs prevent cancer from occurring in the esophagus in patients with reflux. In fact, while acid suppression by drugs has continued to increase in efficacy, the incidence of adenocarcinoma in the United States and Western Europe has increased over six-fold in the past three decades⁸. Acid suppressive drugs are not effective in preventing cancer. The use of acid suppressive drugs as the only drug treatment at present for reflux disease means that an estimated 14,000 people in the USA will develop reflux-induced adenocarcinoma in 2007). Moreover, recent studies suggest that adenocarcinomas presently classified as proximal gastric carcinoma (adenocarcinoma of the gastric cardia) are also adenocarcinomas of the distal reflux-damaged esophagus¹⁰. This adds another approximately 10,000 cases to the number of reflux-induced adenocarcinomas in the USA, making the annual expected number of cases 24,000 in 2007.

Present drug treatment has no effect in preventing these cancers. Anti-reflux surgery has the potential to prevent cancer but is unproven and is unlikely to be performed in adequate numbers or with adequate effectiveness to impact this cancer incidence.

Treatment Methodologies

The current invention is directed to treatment methodologies that treat reflux carditis in accordance with this novel mechanistic understanding of the disorder. Specifically, the current invention is directed to treatment methodologies that manipulate the two early biological metaplastic events (i.e., the cardiac mucosal shift to oxyntocardiac mucosa or intestinal metaplasia) to reverse the genetic switches in a manner that would prevent the progression to adenocarcinoma. Even more specifically, the current invention is directed to medications that prevent esophageal adenocarcinoma by treating the metaplastic reactions whereby columnar epithelium is converted to cardiac mucosa and cardiac mucosa converts to either oxyntocardiac or cardiac mucosa.

There is presently no research into these two biologic reactions because there is no recognition of these early changes of reflux disease. At the present time reflux disease is diagnosed when erosive esophagitis is present. When erosive esophagitis is absent and no columnar lined esophagus is visualized endoscopically, the patient with classical reflux symptoms is designated as having non-erosive reflux disease (NERD). Simply put there is presently no recognized diagnosis between erosive esophagitis and Barrett's esophagus in the patient with GERD. The finding of cardiac mucosa and oxyntocardiac mucosa in the esophagus is interpreted as “normal gastric mucosa” in the esophagus. In short, the reason why no research is directed at these two biological reactions at the present time is that they are not recognized as abnormal and early steps in the sequence of changes whereby reflux causes cancer. The deficiencies of the current protocols are discussed in detail, below.

Deficiencies in Current Treatment & Diagnostic Protocols

FIG. 9 shows illustrations of four patients with different and constant levels of reflux. First is a patient without reflux, as shown in FIG. 9A, the patient has only squamous epithelium (gray) and gastric oxyntic mucosa (yellow). FIG. 9B shows a patient with mild reflux (column of reflux shown as an orange triangle), as illustrate the patient has a short segment of metaplastic columnar epithelium (black), shown here to be limited to the dilated end-stage esophagus. In a patient with moderate reflux (FIG. 9c) there is a longer segment of columnar lined esophagus, extending into the tubular esophagus from the dilated end-stage esophagus. Finally, a patient with severe reflux (FIG. 9D) has a long segment of columnar lined esophagus.

As shown at the bottom of FIG. 9, most patients show a progressive worsening of the severity of reflux with age. The columnar metaplasia progressively increases in length. However, the initial several decades of this change contains only cardiac and oxyntocardiac mucosa. As shown, the first metaplasia from squamous epithelium to cardiac mucosa can precede cancer by several decades. The detection of this first metaplastic change is easily achieved by endoscopy and biopsy. Once detected, there is a window of opportunity for manipulating cardiac mucosa and its evolutionary epithelia that is often 20-40 years long. This window of opportunity is ignored at present because of the failure to recognize these biological reactions as early cellular changes of reflux disease.

Indeed, at the present time, the first time the patient is regarded as having a cancer risk is when Barrett's esophagus develops. There is no attempt made to prevent cardiac mucosa from evolving into intestinal metaplasia. There is no attempt made to promote conversion of cardiac mucosa to oxyntocardiac mucosa. In fact, these manipulations are not the target of any research at this time. It is to these reactions that the treatments of the current invention are directed.

Treatment Evaluation

The current invention is directed to early interventions that will prevent the progression of the patient in the reflux to adenocarcinoma sequence. First, there needs to be a standardized methodology to evaluate the effectiveness of the current treatment regimes.

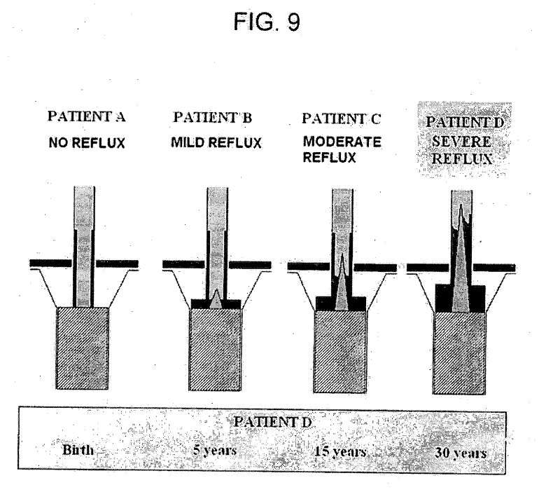

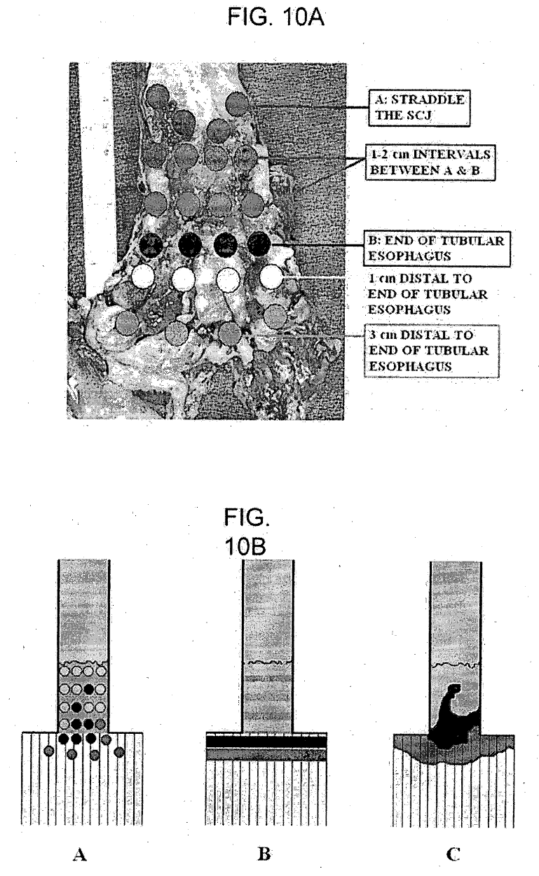

In one embodiment, shown in FIG. 10, a standardized biopsy protocol in patients with columnar lined esophagus is proposed. In the methodology, four quadrant biopsies are taken at 1-2 cm intervals in the tubular esophagus as well as retrograde biopsies distal to the tubular esophagus. The different columnar epithelial types in each biopsy are then defined histologically to produce an accurate map of the three different epithelial types within the columnar lined segment of esophagus (blue=intestinal metaplasia; black=cardiac mucosa; red=oxyntocardiac mucosa). FIGS. 10A to 10C show schematics of three ways of mapping the columnar epithelia.

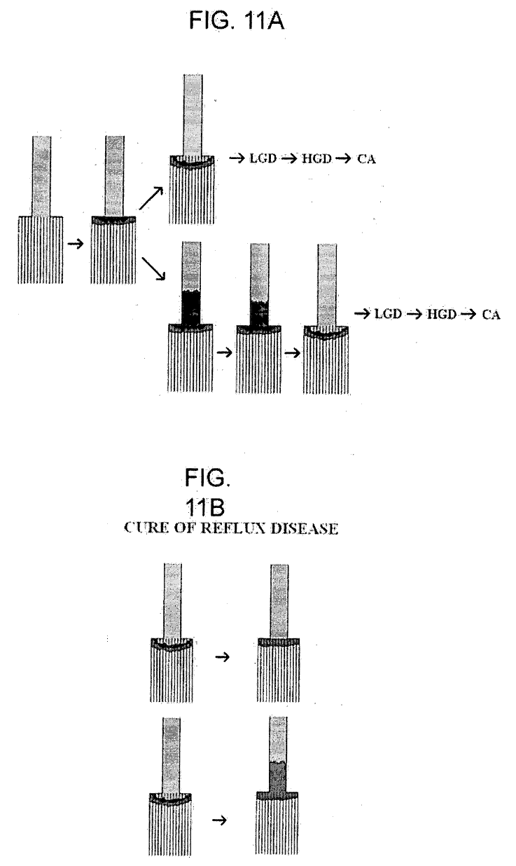

Biopsies taken at standard endoscopy would also be effective in detecting the presence of oxyntocardiac and intestinal epithelia within the columnar lined esophagus. As such, the effectiveness of any drug in causing changes in the metaplastic sequence can be readily followed with sequential biopsy. The mapping of the different types of columnar lined esophagus by a standardized biopsy protocol will therefore permit detection of diminished risk of cancer if intestinal metaplasia is converted to cardiac mucosa and cardiac mucosa converted to oxyntocardiac mucosa, as shown in FIG. 11, which shows the progression of reflux-induced columnar epithelium to adenocarcinoma through cardiac mucosa (black) and intestinal metaplasia (blue). This is the course in all patients who progress from reflux to esophageal adenocarcinoma. Regression and “cure” induced by the drugs of the current invention results in conversion of all cardiac and intestinal epithelia into oxyntocardiac mucosa (red). This is an epithelium that is not at risk for progressing to adenocarcinoma

This conversion to oxyntocardiac mucosa represents a new definition for regression or cure of columnar metaplasia. At the present time, the only regression or cure of Barrett esophagus that is recognized is the replacement of columnar metaplasia by squamous epithelium and the disappearance of intestinal metaplasia. No authority at the present time recognizes the conversion to oxyntocardiac mucosa as having any importance.

Novel Pharmaceuticals for Preventing Adenocarcinoma

The present invention is directed generally to novel medications for the prevention and treatment of gastroesophageal reflux disease, and more specifically to medications that operate in accordance with a new mechanistic understanding of gastroesophageal reflux disease. Specifically, the following manipulations involving these early biological reactions would result in a significant if not absolute decrease in the cancer risk in patients with reflux disease.

Inhibiting Conversion Of Cardiac Mucosa To Intestinal Metaplasia

In one embodiment, the current invention is directed to a treatment methodology for preventing intestinal metaplasia by inhibiting the activation of the cardiac to intestinal genetic switch by targeting the refluxate responsible for the turning on the genetic switch and inactivating it, combining with it and thereby rendering it ineffective, or neutralizing its action. In this embodiment, cancer is prevented because cardiac mucosa is prevented from progressing to intestinal metaplasia. As previously discussed, it is only the intestinal metaplasia that is susceptible to carcinogenesis (See, e.g., the discussion related to FIG. 4).

In one preferred embodiment, the treatment methodology of the current invention is direct to inactivating bile salt derivatives that reach the stomach via duodenogastric reflux. In such an embodiment, a treatment medication could operation in any one the following ways:
(a) In one embodiment the drug would operate by altering the gastric milieu that promotes the generation of the specific bile salt derivative involved in the switch from bile acids. For example, if the specific molecule is produced from bile acids at a specific pH, change in the gastric pH milieu can cause the molecule responsible not to be formed in the stomach, thereby preventing intestinal metaplasia in the esophagus when reflux occurs.
(b) In another embodiment the drug would operate by inhibiting and destroying the molecule itself; and
(c) In a final embodiment, exogenous chemicals that prevent the conversion of cardiac mucosa to intestinal metaplasia could also be used.

Although treatments involving a few specific refluxate molecular triggers are described above, it should be understood that other refluxates may be targeted by the current treatment methodology. Accordingly, in another embodiment the current invention is directed to a drug discovery methodology. In this embodiment the refluxate molecule involved in triggering the genetic switch is identified by growing cardiac epithelial tissue of cardiac stem cell lines in tissue culture and testing the effect of various chemicals in this experimental system for evidence of intestinal metaplasia. The end-point of the system will be the detection of goblet cells, antigens and mucins associated with goblet cell differentiation, or the detection of expression of the genetic switch associated with intestinal metaplasia.

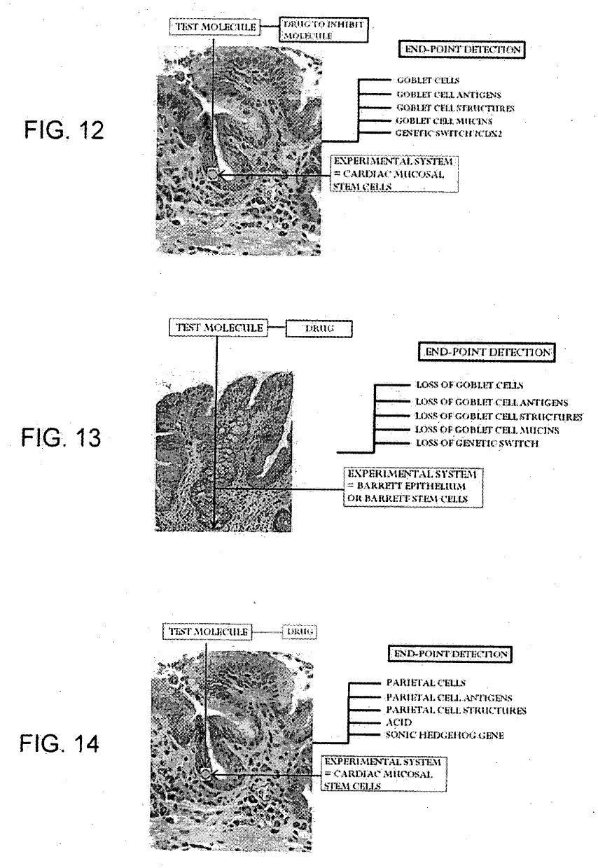

FIG. 12 shows a drug discovery methodology that could be utilized to detect the conversion of cardiac epithelial cells to intestinal metaplasia in vitro. As shown, the test base would be cardiac mucosa. This could either be strips of cardiac mucosa grown in tissue culture or cardiac mucosal stem cell lines that are established. This mucosa would be tested against the test molecule/s of choice. For example, in one embodiment these molecules would be components of the gastric juice. The end-point of a positive test would be the detection of the genetic switch (e.g., CDX2), detection of cellular structural elements of goblet cell differentiation, detection of antigens specifically associated with goblet cell differentiation, and detection of specific mucin types associated with goblet cells. The drug could then be tested to determine its ability to inactivate, neutralize or inhibit the action of this molecule in gastric juice.

The detection of such molecules will require an experimental system that consistently causes intestinal metaplasia to develop in the cardiac epithelia; tissue culture system. Once this is in place, many different target molecules can be tested for efficacy in preventing intestinal metaplasia from taking place, with appropriate controls to show that intestinal metaplasia would occur if the molecule/drug was not present.

Because intestinal metaplasia occurs in humans with cardiac mucosa in the natural state of progression of the disease, the search for the molecule in question can be optimized by looking at the components of gastric juice in patients who have developed intestinal metaplasia.

Regardless of the specific treatment drug, because there is a long window of time before cardiac mucosa transforms to intestinal metaplasia, such intervention is feasible (see, e.g., discussion regarding FIG. 9). As previously discussed, the presence of cardiac mucosa in the patient can be detected by endoscopy and biopsy many years prior to the development of intestinal metaplasia (FIG. 10). The use of the medications of the current invention at any time during the many decades during which the patient has non-intestinalized cardiac mucosa would serve to prevent intestinal metaplasia and cancer. However, as long as cardiac mucosa persists, it is likely that the drug will have to be used on a long-term and possible life-long basis.

Promoting Conversion Of Intestinal Metaplasia To Cardiac Mucosa

In another embodiment, the current invention is directed to a treatment methodology for preventing cancer by reversing the genetic switch that results in intestinal metaplasia of cardiac mucosa. In such an embodiment the treatment will be directed at stem cells from intestinal metaplastic epithelium. Again, a successful treatment will be indicated by either a decrease in goblet cells, villin, the acid mucin that is typical of intestinal metaplasia, the loss of antigens associated with intestinal metaplasia, and the loss of the genetic switch that induces intestinal metaplasia.

The current invention is also directed to drug discovery methodologies for developing medications that would promote the conversion of intestinal metaplasia to cardiac mucosa. One embodiment of such a system is provided in FIG. 13, which shows an experimental system that could be utilized to detect the conversion of intestinal metaplastic epithelium to cardiac mucosa in vitro. As shown, in such a system the test base would be intestinal mucosa. This could either be strips of intestinal (Barrett's) mucosa grown in tissue culture or intestinal metaplastic stem cell lines that are established (non-dysplastic Barrett's epithelium cell lines are available commercially). This would be tested against a variety of test molecules.

As described above, the end-point of a positive test would be the loss of the genetic switch responsible for goblet cell metaplasia (believed to be CDX2 at the present time), loss of cellular structural elements of goblet cell differentiation, loss of antigens specifically associated with goblet cell differentiation, and loss of specific mucin types associated with goblet cells. The molecules would then be tested in humans with biopsy proven Barrett's esophagus to see whether it reproduces the reversal of intestinal metaplasia to cardiac mucosa in vivo. If all intestinal metaplasia is reversed in the patient, the drug would remove all risk of adenocarcinoma in the patient.

Promoting Conversion Of Cardiac Mucosa To Oxyntocardiac Mucosa

In another embodiment, the invention is directed to a treatment methodology to prevent cancer by promoting the genetic switch that causes cardiac mucosa to transform into oxyntocardiac mucosa. In such an embodiment, the esophageal epithelium would be converted to a columnar epithelial type that is stable, resists acid damage, does not cause pain when exposed to acid, and does not progress to cancer.

Suitable molecules that that could cause the conversion of cardiac to oxyntocardiac mucosa include: (a) strong acids like hydrochloric acid, bromic acids; (b) weak acids like acetic acid and oxalic acids; (c) other anions such as Sodium, Potassium and Lithium; (d) other non-acid molecules in the refluxate such as pepsin, bile acid components, mucopolysaccharides; and (e) exogenous molecules that produce the same effect as the refluxate molecule that produces this genetic switch in vivo.

Although some specific treatments are proposed above, the current invention is also directed to a drug discovery methodology for developing additional treatments for promoting the conversion of cardiac mucosa to oxyntocardiac mucosa. In one such embodiment, medications may be developed by evaluating the effect of various chemicals on tissue culture systems with cardiac mucosa or stem cells from cardiac mucosa. The linkage of the expression of Sonic Hedgehog gene and oxyntocardiac mucosa provides a model for research testing of cardiac mucosal stem cells with different compounds. The detection of the expression of the new gene will be the end point for a successful conversion of cardiac to oxyntocardiac mucosa in an experimental cell system. Other events that would indicate conversion of cardiac to oxyntocardiac mucosa in an experimental system would be the appearance of parietal cells, secretion of acid and the appearance of new ultrastructural and antigenic elements associated with parietal cells.

One exemplary drug discovery system is shown in FIG. 14, which illustrates a system that would be utilized to detect the conversion of cardiac to oxyntocardiac mucosa in vitro. In such a system the test base would be cardiac mucosa. This could either be strips of cardiac mucosa grown in tissue culture or cardiac mucosal stem cell lines that are established. This mucosa would be tested against a variety of test molecules. As discussed above, the end-point of a positive test would be the detection of the genetic switch (believed to be Sonic Hedgehog gene at the present time), detection of cellular structural elements of parietal cell differentiation, detection of antigens specifically associated with parietal cell differentiation, and detection of acid secretion by the parietal cell that is produced. Any molecule that is found to cause this change in vitro would be a potential cancer preventing drug that can be tested in human systems.

Once developed, this drug can be tested in patients who have cardiac mucosa with and without intestinal metaplasia, confirmed by endoscopy and biopsy using the standardized biopsy protocol shown in FIG. 10. The drug is likely to be effective when used at the stage when the patient with reflux has only cardiac mucosa. At a later stage when intestinal metaplasia has developed, it may be necessary to first convert the intestinal metaplasia to cardiac mucosa (see above) before this drug becomes effective. Because there is a long lag phase of many decades between development of cardiac mucosa and intestinal metaplasia, it is feasible to identify the patient with only cardiac mucosa.

Regardless of the actual treatment used, conversion of all cardiac and intestinal mucosa in the esophagus to oxyntocardiac mucosa will result in a stable epithelium that is resistant to cancer. Accordingly, this treatment methodology will be capable of cancer prevention as it completes the change in epithelium. In this embodiment, it would not be necessary to continue using the drug unless the oxyntocardiac mucosa reverts to cardiac mucosa or new cardiac mucosa arises from continued damage of squamous epithelium.

A Methodology For The Acidification Of The Esophagus

It has been shown in the above discussion that the pH gradient set up in the esophagus in the patient with reflux disease influences the changes in esophageal epithelia. (See discussion of FIGS. 7 & 8.) Specifically, the conversion of cardiac mucosa to intestinal metaplasia in vivo appears to be highly dependent upon the pH milieu of the esophagus. The acid, which damages the squamous epithelium causes symptoms, erosions and induces columnar metaplasia. As columnar metaplasia increases in length, the squamous epithelium progressively moves further up the esophagus to a less acid part of the gradient and damage becomes less. The columnar metaplastic epithelium that separates squamous epithelium from the stomach is exposed to a higher acid level in the reflux-induced gradient, which protects against progression to intestinal metaplasia and cancer. This conversion is favored by a pH milieu in the 4-6 range and appears to be inhibited by a strong acid pH. In a strong acid milieu, the columnar epithelium tends towards oxyntocardiac mucosa and away from intestinal metaplasia. Accordingly, acidification of the esophagus that has columnar metaplasia therefore has the potential to prevent intestinal metaplasia in cardiac mucosa.

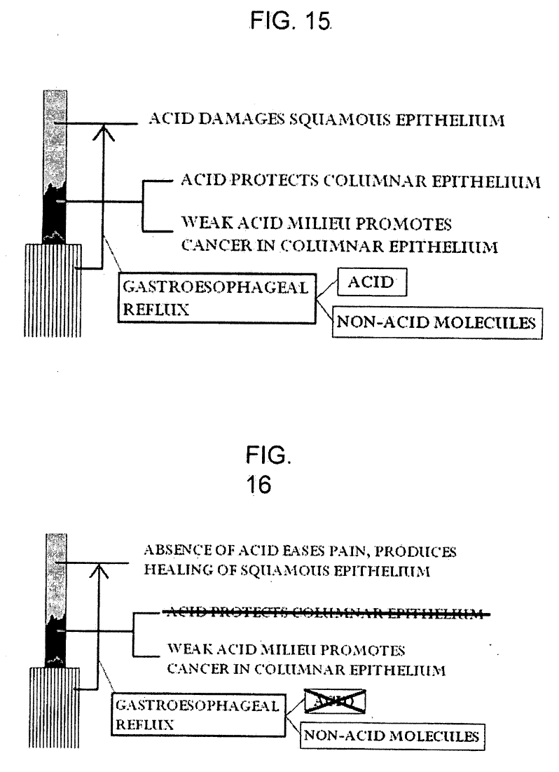

FIG. 15 illustrates the effects of the pH gradient created by gastroesophageal reflux in different components of the esophageal epithelium. As shown, strong acid damages the squamous epithelium but tends to prevent cancer in columnar epithelia by causing it to differentiate toward oxyntocardiac mucosa and away from intestinal metaplasia. A weak acid milieu is effective in preventing squamous epithelial damage, but promotes adenocarcinoma by causing the columnar epithelium to differentiate away from oxyntocardiac mucosa and towards intestinal metaplasia.

Accordingly, altering the pH milieu of the esophagus by decreasing the pH to the strong acid range (pH 1-2) throughout the area that has intestinal metaplasia is likely to be effective in causing intestinal metaplasia to revert to cardiac mucosa. Moreover, altering the pH milieu of the esophagus by decreasing the pH to the strong acid range (pH 1-2) throughout the area that has columnar lining is predicted to promote the conversion of cardiac mucosa to oxyntocardiac mucosa. This is what happens in the natural state in patients with GERD where the cardiac mucosa in the more distal esophagus, which is subjected to a strong acid milieu during episodes of reflux, commonly transform into oxyntocardiac mucosa.

Although previously discussed, it is to be emphasized that the presence of oxyntocardiac mucosa converts the esophageal epithelium into a stable and benign epithelium that does not progress in the reflux to adenocarcinoma sequence. Accordingly, in one embodiment the current invention is directed to a treatment methodology directed at preventing esophageal cancer by acidification of the esophagus of patient's with columnar metaplasia.

Acid suppressive drug therapy, as used presently in patients with reflux, decreases the acidity of the entire esophageal segment. The increase in pH to the 4-7 range protects the squamous epithelium, relieving pain and healing erosions, the effect that is sought by these drugs; however, the low acidity in the esophagus removes the protection provided by strong acid to the columnar epithelia, causing them to progress away from oxyntocardiac mucosa and towards intestinal metaplasia, promoting cancer.

FIG. 16 illustrates the pH changes induced in the esophagus with acid suppression. As shown, the drugs act by decreasing acidity in the gastric refluxate. The weaker acid (pH 4-6) milieu in the esophagus heals the damaged squamous epithelium, relieving pain and healing erosions. However, the weak acid environment promotes intestinal metaplasia and cancer in columnar epithelium. As such, it has been surprisingly discovered that acid suppressive drugs promote adenocarcinoma, and are at least partly responsible for the increased incidence of reflux-induced cancer that has been seen in the past four decades.

Accordingly, one embodiment of the invention is directed to a treatment methodology for altering the usual pH gradient in the esophagus seen in the patient with reflux. However, creating an environment that is strongly acid (pH 1-2) throughout the entire length of the esophagus is simply not practical or desirable. Such a treatment methodology would cause:
(a) Squamous epithelial damage causing pain, increased permeability, erosions, ulcerations and strictures;
(b) Inhibition of the conversion of cardiac mucosa to intestinal metaplasia, a change that will prevent progression to adenocarcinoma;
(c) Reversal of non-dysplastic intestinal metaplasia already present to cardiac mucosa, a change that will prevent progression to adenocarcinoma; and
(d) Promotion of the conversion of cardiac mucosa to oxyntocardiac mucosa, a change that will promote progression to adenocarcinoma.

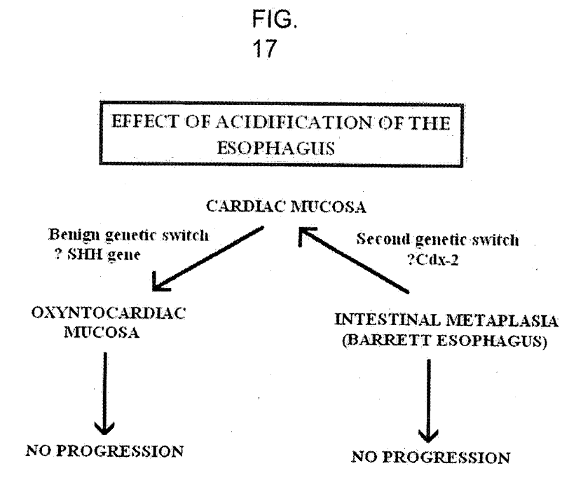

FIG. 17 illustrates the changes expected from strong acidification of the entire segment of the reflux damaged esophagus. (Note: This does not happen in the reflux patient normally because the reflux sets up a pH gradient with progressive decrease in acidity in the more proximal esophagus.) As shown, strong acid exposure of columnar epithelium would cause intestinal metaplasia to switch back to cardiac mucosa and cardiac mucosa to switch to oxyntocardiac mucosa. These switches will stop the progression to adenocarcinoma.

Although such an acidification scheme would prevent adenocarcinoma, to produce a decrease in esophageal pH over the entire extent of columnar lining would also require extreme acidification of the stomach by administration of large amounts of strong acid (e.g., hydrochloric acid). This would mimic the situation of Zollinger-Ellison syndrome. It would also cause a practical problem because the hydrogen ions would tend to severely damage the squamous epithelium of the esophagus in patients with GERD, causing pain, erosions, ulcers and strictures. Such a treatment would also tend to cause peptic ulcers in the stomach and duodenum as seen in Zollinger-Ellison syndrome. Accordingly, simple acidification by giving hydrogen ions in sufficient quantity to produce the required beneficial changes in columnar epithelium is not feasible.

In light of these issues, in one embodiment the current invention is directed to a method of treatment comprising the administration of a complexed form of hydrogen ions that can be delivered into the stomach to permit acidification of gastric juice without causing damage. For example, in one embodiment, the hydrogen ion is combined with a larger inert molecule, such as cellulose, dextran, or some other inert polymer, or it is part of a large organic molecule such as an alkene. In such an embodiment it is possible to produce a drug that has the following characteristics: (a) it will be too large to penetrate the squamous epithelium and therefore will not cause damage in the squamous epithelium; (b) it is not injurious to gastric and duodenal mucosa and does not cause peptic ulceration; (c) it is degraded in the duodenum where the H+ ions will be neutralized and the inert particle either degraded or remain in the intestine without being absorbed; and (d) it is capable of reacting with columnar epithelia of the esophagus to produce the same biologic changes as if it was acid.

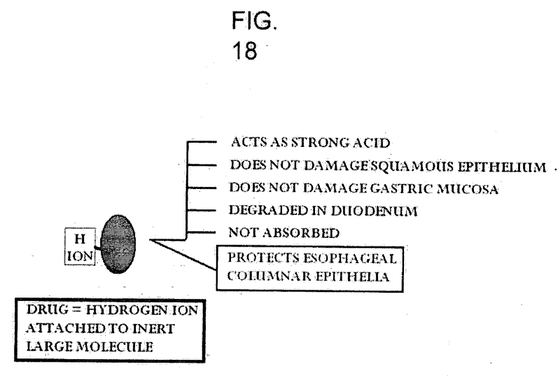

FIG. 18 provides an illustration of a model medication to prevent reflux-induced esophageal adenocarcinoma by the targeted delivery of H+ ions. As shown, the drug has active hydrogen ion that acidifies the esophagus but, because of its attachment to the inert moiety, has a size that prevents it from entering and therefore exerting a damaging effect on the squamous epithelium, thereby preserving the protective action of acid on esophageal columnar epithelia. In this regard, it must be recognized that the foveolar pit of metaplastic columnar epithelium is much wider by a factor of several hundred times than the intercellular spaces between squamous epithelial cells. There is, therefore, a large window wherein the drug can be made accessible to the stem cells of columnar epithelium but not penetrate the squamous epithelium to reach the stem cell in its basal layer.

The nature of the inert component of the drug will determine its size as well as its tendency to be absorbed and its susceptibility to degradation by the enzymes of the gastrointestinal tract. In this regard, cellulose, which is not digested in the gastrointestinal tract, appears attractive. It would also add fiber to the diet that may have a secondary beneficial effect. Dextran, various alkenes, and artificial polymers of other types have the advantage of infinite manipulation with regard to size and molecular shape that also make them attractive.

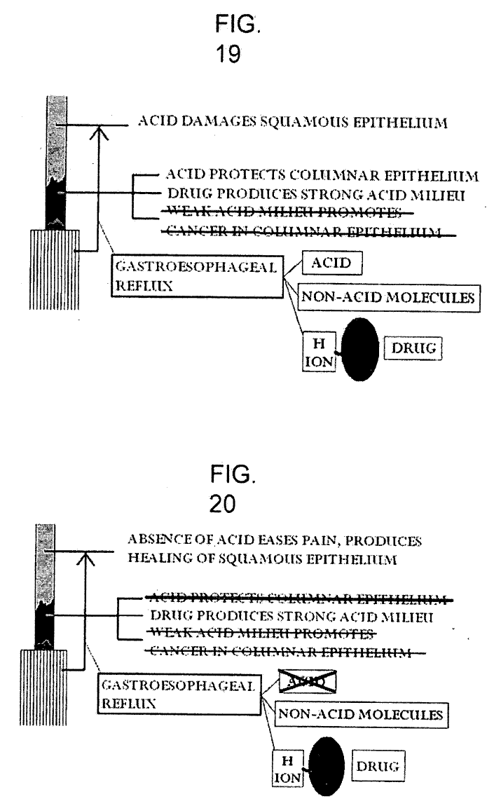

As shown in FIG. 19, administration of the medication of the current invention orally in sufficient dosage to produce an adequate concentration in gastric juice would result in the medication being delivered to the entire length of affected esophagus when the patient has episodes of gastroesophageal reflux. The medication would remove the weak acid milieu that is present in the esophagus as a result of the natural pH gradient created by the patient's reflux. In turn, by maintaining the pH changing potential of the H+ ion, this medication would have the same effect as strong acidification on the columnar epithelia of the esophagus, causing these columnar epithelial to progress away from intestinal metaplasia and towards oxyntocardiac mucosa, thereby decreasing cancer risk.

In another embodiment, this of H+ ion delivery medication could be combined with acid suppression to inhibit the patient's native acid secretion. This would permit the manipulation of the columnar epithelium with the new drug while using the positive effect of acid suppression in controlling squamous epithelial damage, pain, erosions as occurs at present. FIG. 20 provides an illustration of the use of medications in combination with acid suppressive drugs. As shown, the removal of the patient's native acid by acid suppressive drugs has the desired protective effect on the squamous epithelium in easing symptoms and healing erosions. The medication in turn would create the strong acid milieu throughout the length of the esophagus that protects columnar epithelia and preventing progression to intestinal metaplasia and cancer. This combination treatment medication will provide the hydrogen ion analogue to the columnar epithelium that will negate the alkalinity resulting from the use of acid suppressive drugs alone. In summary, the cancer-promoting effect of acid suppression is therefore removed while its therapeutic effect is maintained.

Medication Delivery and Testinq

As discussed above, the current invention is directed to both treatment methodologies and drug discovery methodologies. Although not all possible treatment medications are disclosed herein, as discussed above identification of chemicals that can influence the biologic targets set forth in the current application is with present technology. To this end the current application discloses exemplary drug discovery systems using cardiac and intestinal epithelia in tissue culture. In addition, cell lines are commercially available for intestinal metaplastic epithelia. Finally, the current invention also provides a circumscribe set of refluxate targets upon which a medication would be expected to work. Specifically, the conversions of cardiac to intestinal metaplasia and oxyntocardiac mucosa occur in vivo in patients with GERD, as such the molecules that cause these changes must be present in gastric refluxate in patients who develop columnar metaplasia of the esophagus. This limits the search for the chemicals producing these biological reactions. Moreover, effective end-points from detection of the changes in these systems are present and have been described in the relevant sections above. Using these drug discovery systems and end-points will permit effective research for additional medications in a straight-forward manner.

Meanwhile, the treatment methodology involving acidifying the esophagus using a hydrogen ion complex has also been described. Again, although specific compounds have been described, many other molecular combinations can be contemplated by the current invention. Acidification uses the fact that the molecules responsible for the genetic switches are present in the gastric refluxate of patients with reflux and simply uses the fact that the effect of these molecules are influenced by the pH milieu in the esophagus. Accordingly, manipulation of the esophageal pH towards stronger acidity than created by natural reflux with the use of the new medication will cause columnar epithelia to move away from intestinal metaplasia and towards oxyntocardiac mucosa. As such, any medication that operates in a manner opposite to the presently used acid suppressive drugs, which decrease the acidity in the esophagus and promote intestinal metaplasia and adenocarcinoma, may be use in accordance with the treatment methodology of the current invention.

Delivery of Drugs To The Target Site

Normally, insuring that a drug is delivered in an effective dose to the correct location is a significant challenge. However, as described above reflux disease, is a unique disorder with unique opportunities for intervention. The target cells are damaged by the episodic reflux that occurs in the patient. In the evolution of cellular changes, some of these changes are deleterious and others are actually protective. For example, conversion of cardiac mucosa to oxyntocardiac mucosa is a protective change—the disease itself provides the protection naturally because it is the reflux that causes the conversion of cardiac to oxyntocardiac mucosa.

As a result of these inherent properties, any drug that is developed in accordance with the current invention will be effectively delivered to the target cell in the esophagus by the patient's disease itself. Indeed, all that needs to be done is to give the drug in sufficient dosage orally to develop an adequate concentration in the stomach. Then, when the patient has a reflux episode, the drug will be delivered to the target cell in the esophagus and then cleared back into the stomach. In short, the drug will utilize the same delivery system as the disease uses to deliver molecules in the refluxate to the target cell.

As a result of this natural drug delivery, the medications in accordance with the current invention may be delivered by almost any suitable means, such as, for example, via a pill or liquid administered orally at sufficient frequency and in sufficient concentration. Regardless of the delivery system chosen, the medication should be designed to go into solution and remain inert within the stomach such that it effectively becomes part of the gastric juice. In turn, the drug will be delivered to the esophageal stem cells by gastroesophageal reflux. It has been shown that the amount of columnar metaplasia that is present in the esophagus is specifically related to the severity of reflux, as such the delivery of the drug to the stem cells in metaplastic columnar epithelial in the esophagus will be controlled to the appropriate level by the severity of the reflux in the patient.

Again, because of the nature of the disorder itself there is no need to develop a delivery system for the drug because the disease serves as the delivery system. The only requirement is that the concentration of the drug in gastric juice must be high enough that the drug will be able to exert its positive effect during the short exposure of the target cells to refluxate before the esophagus clears the refluxate. Although only one metaplastic change is discussed above, it should be recognized that every metaplastic change and genetic change that causes cancer is also subject to the same requirement.

Testing Drug Efficacy

There are easy and effective methods for testing any drugs that are developed. Patients with cardiac mucosa are found in the population in large numbers. It is expected that 40-50% (80-100 million people) of the adult population in the USA has cardiac mucosa, some with GERD symptoms but some without. Indeed, it is believed that 5-15% (10-30 million people) of the adult population has intestinal metaplasia (Barrett esophagus) in the USA, some with symptoms of GERD but some without. Identification of these patients is easy based on standard upper GI endoscopy with a standardized biopsy protocol. Once identified, these patients can be randomized into a control and test group to test the efficacy of any new drug. Because the changes in the metaplastic types occur over a short time frame, it is anticipated that the drugs tested will produce the change quickly. The change can be detected by repeat endoscopy with biopsies taken with the same standardized protocol. Changes in the different types of the columnar epithelial will in turn determine drug effectiveness.

Possible Side Effects

The drugs that are envisaged have an action that is limited to the mucosa of the esophagus. However, it is necessary that the drugs reach a sufficient concentration in gastric juice because the delivery of the drug to the target epithelium is by gastroesophageal reflux, because of gastric emptying, the concentration of the drug in the stomach will fall off over the usual 6-8 hours of gastric emptying, necessitating repeated oral doses to maintain gastric concentration. Because the drug has no necessity beyond the stomach, it can be designed to be inactive beyond its point of required activity. Accordingly, in one embodiment of the invention the medication is designed to be degraded in the first part of the duodenum to inactive, unabsorbed components.

In such an embodiment, inactivation would occur by the dissociation of the hydrogen ions from the inert moiety. Under these circumstances, the hydrogen ions will be immediately neutralized by the alkaline duodenal contents. This will prevent changes in the remainder of the intestine as well as limit systemic toxicity because no component of the drug will ever enter the systemic circulation. Because there is no evidence that strong acidity is deleterious to gastric mucosa, which is designed to withstand acid normally, the drug should have no side effects on the gastric mucosa. This is particularly true if the hydrogen ions remain attached to the inert moiety until the drug reaches the duodenum. The drug can therefore be designed to have its activity limited to the epithelia of the esophagus. Using such a methodology, the efficacy of the drug in reducing esophageal adenocarcinoma will be so specific in its action that it will not be necessarily associated with effects in any other cell of the body.

### SUMMARY

Regardless of the treatment regime chosen, the first crucial step identified by the current invention is the understanding of the cellular processes involved. The failure to do this in reflux disease has resulted in much misdirected research. By identifying the molecular mechanism that converts cardiac mucosa to oxyntocardiac mucosa it becomes possible to induce this change through the treatment regimes of the current invention. These treatment methodologies fall into two broad categories and include:
administering a medication to induce oxyntocardiac mucosa; and
administering a medication to inhibit or reverse the molecular step involved in the conversion of cardiac mucosa to intestinal metaplasia.

### CONCLUSION

The novel gastroesophageal reflux disease treatment methodologies of the current invention are superior to any conventional measure. Moreover, because the main complication of reflux disease is adenocarcinoma, the current invention also relates to the prevention of this advanced form of the disease.

While preferred embodiments of the foregoing invention have been set forth for purposes of illustration, the foregoing description should not be deemed a limitation of the invention herein. Accordingly, various modifications, adaptations and alternatives may occur to one skilled in the art without departing from the spirit and scope of the present invention.

What is claimed is:

1. A method of treating gastroesophageal reflux disease in a patient comprising:
providing a therapeutic course of treatment to prevent or reverse a disorder that triggers transformation of a healthy esophageal epithelium to an unhealthy esophageal epithelium.

2. The method of claim 1, wherein the healthy epithelium is a squamous epithelium.

3. The method of claim 1, wherein the unhealthy epithelium is a metaplastic columnar epithelium that includes cardiac mucosa and intestinal metaplasia.

4. The method of claim 1, wherein the treatment comprises suppressing at least one class of molecule that promotes the transformation of cardiac mucosa to intestinal metaplasia.

5. The method of claim 4, wherein the at least one class of molecule is a naturally occurring molecule in the gastroesophageal refluxate.

6. The method of claim 5, wherein the molecule in the refluxate includes a bile salt derivative.

7. The method of claim 6, wherein the bile salt derivative is selected from the group consisting of: cholic acid and dehydrocholic acid.

8. The method of claim 4, wherein the step of suppressing includes lowering the pH of the patient's gastric juice.

9. The method of claim 8, wherein the pH of the patient's gastric juice is maintained in a range of between about 1 to 3 and is delivered by a reflux episode to the unhealthy columnar epithelium.

10. The method of claim 8, wherein the treatment includes administering an agent to lower the pH of the environment of the unhealthy esophageal columnar epithelium without causing damage to the healthy esophageal squamous epithelium.

11. The method of claim 10, wherein the agent comprises a hydrogen ion complexed with an inert molecule.

12. The method of claim 11, wherein the inert molecule is sized such that the hydrogen ion complex is capable of penetrating the foveolar pit of a metaplastic columnar epithelium, but cannot penetrate the intercellular spaces of a squamous epithelium.

13. The method of claim 12, wherein the inert molecule is one of either an inert polymer or an inert organic molecule.

14. The method of claim 11, wherein the inert molecule is selected from the group consisting of cellulose, dextran and alkenes.

15. The method of claim 8, further comprising administering an acid suppressive agent to suppress the native acid secretion by the patient.

16. The method of claim 5, wherein the step of suppressing includes administering an agent to inhibit the refluxate molecule responsible for converting cardiac mucosa to intestinal metaplasia.

17. The method of claim 1, wherein the treatment comprises a therapy to promote conversion of cardiac mucosa to an oxynto-cardiac mucosa epithelium.

18. The method of claim 17, wherein the therapy includes a therapy to promote the genetic switch that induces the production of parietal cells in the cardiac mucosa.

19. The method of claim 18, wherein the genetic switch involves the Sonic Hedgehog gene.

20. The method of claim 18, wherein the therapy includes administering an agent selected from the group consisting of strong acids, weak acids, anions, and non-acid refluxate molecules.

21. The method of claim 17, wherein the treatment further comprises a therapy to first promote the conversion of intestinal metaplasia to cardiac mucosa in that group of patients that have intestinal metaplasia at the time of diagnosis.

22. The method of claim 21, wherein the treatment further includes a preliminary therapy to reverse the genetic switch that induces the production of intestinal metaplasia in cardiac mucosa.

23. The method of claim 22, wherein the genetic switch involves the CDX2 gene.

24. The method of claim 1, wherein the therapeutic course is delivered orally at a sufficient concentration to ensure delivery to the effected area during gastroesophageal reflux.

25. The method of claim 1, wherein the therapeutic course is administered in a form that is prevented from being absorbed into the circulation and is inactivated in the duodenum.

26. A method of screening therapeutic compounds effective for treating gastroesophageal reflux disease, comprising:
exposing at least one sample of epithelium to a plurality of molecular components in gastric juice;
monitoring said at least one sample for the different types of columnar cellular transformation.

27. The method of claim 26, wherein the cellular transformation monitored is selected from the group consisting of cardiac mucosa to intestinal metaplasia, intestinal metaplasia to cardiac mucosa, and cardiac mucosa to oxynto-cardiac mucosa.

28. The method of claim 26, wherein the epithelium is one of either an epithelium grown in a tissue culture or an established epithelial stem cell line.

29. The method of claim 28, wherein the epithelium is selected from the group consisting of cardiac mucosa and intestinal metaplastic cells,

30. The method of claim 27, wherein the transformation to be detected is from cardiac mucosa to intestinal metaplasia and wherein the step of monitoring includes testing for an end-point selected from the group consisting of detection of the genetic switch responsible for goblet cell differentiation, detection of cellular structural elements of goblet cell differentiation, detection of antigens associated with goblet cell differentiation, and detection of specific mucin types associated with goblet cells.

31. The method of claim 27, wherein the transformation to be detected is from intestinal metaplasia to cardiac mucosa and wherein the step of monitoring includes testing for an end-point selected from the group consisting of detection of loss of the genetic switch responsible for goblet cell differentiation, detection of a loss of cellular structural elements of goblet cell differentiation, detection of a loss of antigens associated with goblet cell differentiation, and detection of a loss of specific mucin types associated with goblet cells.

32. The method of claim 27, wherein the transformation to be detected is from cardiac mucosa to oxyntocardiac mucosa and wherein the step of monitoring includes testing for an end-point selected from the group consisting of detection of the Sonic Hedgehog genetic switch, detection of parietal cells, detection of the secretion of acid and the presence of ultrastructural and antigenic elements associated with parietal cells.

33. The method of claim 26, further comprising comparing the activated genes in the transformed sample to determine the identity of the gene activated in the metaplastic switch.

34. The method of claim 26, further comprising screening at least two epithelial samples taken from patients at high and low risk for carcinogenesis and comparing the results.
